(19) United States
(12) Patent Application Publication
(10) Pub. No.: US 2022/0313706 A1
(43) Pub. Date: Oct. 6, 2022

(54) COMPOSITION COMPRISING STEROL DERIVATIVES FOR USE IN THE TREATMENT OF A NEURONAL PATHOLOGY RELATED TO HYPOXIA, HYPOGLYCEMIA AND/OR HYPERGLYCEMIA

(71) Applicant: DENDROGENIX, Liege (BE)

(72) Inventors: Stephane Silvente, Liege (BE); Quentin Marlier, Liege (BE); Arnaud Rives, Liege (BE); Nicolas Caron, Leige (BE)

(21) Appl. No.: 17/631,773

(22) PCT Filed: Jul. 24, 2020
(86) PCT No.: PCT/EP2020/071044

§ 371 Date: Jan. 31, 2022

(30) Foreign Application Priority Data
Aug. 1, 2019 (BE) BE2019/5501

(51) Int. Cl. A61K 31/575; A61K 31/58; A61P 25/28

(52) U.S. Cl. CPC A61K 31/575; A61K 31/58; A61P 25/28

(57) ABSTRACT

A composition comprising sterol derivatives for use in the treatment of a neuronal pathology related to hypoxia, hypoglycemia and/or hyperglycemia affecting cells of the central nervous system.

### TECHNICAL FIELD

The invention relates to the field of neuronal pathologies related to glucose and/or oxygen deprivation. More specifically, the invention relates to a composition comprising compounds derived from sterols of formula (I) or a pharmaceutically acceptable salt of such a compound, for use thereof in the treatment and/or prevention of cerebral ischemia caused by a stroke, a cranial trauma, a cerebral lesion, diabetes-related hypoglycemia, hyperglycemia or a respiratory problem caused in particular by bacterial or viral infection.

### TECHNOLOGICAL BACKGROUND

Ischemia is a decrease in the blood supply to an organ; cerebral ischemia is therefore a decrease in the blood supply to at least one part of the brain. Ischemia consequently causes an interruption at the same time of the supply of oxygen, of the supply of nutrients, such as glucose, and of the elimination of toxic molecules resulting from anerobic metabolism.

Cerebral ischemia is involved in particular in strokes, commonly called “cerebral attacks” and cranial traumas.

Strokes represent the third most common cause of mortality behind cardiovascular diseases and cancer. However, they are the most common causes of serious disability and the second most common causes of dementia throughout the Western world. A disruption of the blood irrigation of the brain is responsible for strokes. Close to 85% of strokes are ischemic in nature, that is to say caused by the obstruction of a blood vessel by a clot, reducing the blood irrigation in a cerebral region. The clot may form locally in a small artery, or may come from the heart or from a lesion of the wall of one of the large cervical arteries. This obstruction thus prevents a sufficient supply of oxygen and metabolites, such as glucose, to meet the metabolic and energy demand of the central nervous system, and causes cerebral ischemia. The consequences are all the more serious because the brain is not able to switch from aerobic metabolism to anaerobic metabolism in order to produce the energy that it requires. Thus, certain cells of the brain can die.

The consequences of strokes depend on many factors: the speed of re-establishment of the blood supply, the duration of the deprivation of oxygen and/or of metabolites, such as glucose, and/or the cerebral location of the stroke. The clinical manifestations result, depending on the area affected, in more or less widespread paralysis, a loss of speech or language, or else a coma, with debilitating after-effect risks.

In addition, respiratory insufficiency due in particular to a bacterial or viral infection can also lead to cerebral ischemia. For example, an ischemic stroke can be triggered when a blood vessel inflammation (vasculitis) or an infection (with herpes simplex virus, for example) narrows the blood vessels which supply the brain. An increasing number of elements also tend to demonstrate that there is, for example, a negative impact of Covid-19 on the nervous system. Various studies have in fact shown that there is a correlation between Covid-19 infection and loss of taste and smell, confusion, headaches, dizzy spells or even stroke, encephalopathy or else myelitis (Ling Mao et al., Neurogical Manifestations of Hospitalized Patients with COVID-19 in Wuhan, China: a retrospective case series study and Neo Poyiadji et al COVID-19-associated Acute Hemorrhagic Necrotizing Encephalopathy: CT and MRI, <doi: https://doi.org/10.1101/2020.02.22.20026500>). The virus in fact appears to be capable of entering the nervous system via the nasal fossae and the olfactory bulb. Covid-19 receptors, which are found in massive amounts in the lungs, are moreover also found at the blood-brain barrier and the nerve endings. Once the virus is in the nervous system, it is possible that it destroys the neurons responsible for correct breathing function. The destruction of the neurons of the autonomic nervous system that are present in the medulla oblongata, leads to and/or worsens respiratory distress in patients. Conversely, it is moreover possible that a low level of oxygen in the blood due to acute respiratory symptoms in patients suffering from Covid-19 is itself responsible for neurological problems. Since neurons are very sensitive to a lack of oxygen, a prolonged and pronounced decrease in the blood oxygen level would impair the neurons, which would end up dying.

Another cause of cerebral ischemia is also cranial trauma which may be benign or serious, with all the possible intermediate states. Its seriousness depends on the existence of intracerebral lesions or the existence of an extracerebral hematoma, bleeding located between the skull and the brain. A cranial trauma may be accompanied by contusions, neuronal lesions, edema, intracerebral hemorrhages and/or ischemia.

Cerebral ischemia is the major threat which weighs on the functional and anatomical outcome of a traumatized brain. It is a diffuse, or multicenter, overall ischemia which affects the cerebral cortex like anoxia or a cardiac arrest would have done. The ischemia affects all of the gray matter, part of the tissues of the central nervous system having the greatest metabolic oxygen and glucose demand. All cerebral functions are thus threatened. Cellular loss may also be massive and marked by visible cerebral atrophy.

The ischemia can take another more local form. The microcirculation of the tissues located around a focus of contusion or of hemorrhage is threatened by vasoconstriction, the effect of a tissue compression, capillary micro thromboses, or else cellular metabolism disorders. Energy production by the cell is compromised since its oxidative metabolism has broken down. Toxic products such as free radicals are released. The slightest respiratory or circulatory event can then precipitate the cells below the viability threshold.

The consequences of cranial traumas can be physical attacks such as paraplegia, hemiplegia, problems with vision, etc. They can also be neuropsychological attacks affecting memory, attention, ability to communicate. Finally, they modify the behavior and personality of the injured individual and the daily life of said individual, and also that of those close to said individual.

Some cases of diabetes present hypoglycemia coupled to hypoxia, the consequences of which are harmful to affected patients.

Hypoglycemia is the most widespread clinical complication in the daily management of diabetics treated with insulin, and continues to be the limiting factor in the glycemic management of diabetes. Severe hypoglycemia affects 40% of diabetics treated with insulin and can lead to cerebral lesions, in particular in the vulnerable neurons of the cortex and of the hippocampus. For example, learning and memory deficiencies are a direct consequence of this neuronal lesion of the hippocampus caused by severe hypoglycemia.

It is known that hyperglycemia is one of the symptoms revealing diabetes. Indeed, diabetes systematically leads to hyperglycemia. In addition, hyperglycemia unrelated to diabetes also exists and the facts in question are varied; for example, the ingestion of foods or beverages containing sugars or the secondary effects of a medicament. Hyperglycemia related or unrelated to diabetes induces harmful effects at the nervous system level.

Furthermore, the combination of diabetes and hyperglycemia worsens the neuronal damage subsequent to other forms of attack on the central nervous system, such as strokes. Several studies demonstrating the relationship between hyperglycemia, diabetes and neuronal damage have been published, in particular Yazi Li et al. (“Autophagy impairment mediated by S-nitrosation of ATG4B leads to neurotoxicity in response to hyperglycemia”, <doi:https://doi.org/10.1080/15548627.2017.1320467>), Ruchi Sharma et al. (“Hyperglycemia Induces Oxidative Stress and Impairs Axonal Transport Rates in Mice”, Published Oct. 18, 2010) and Wenjuan Zhou et al. (“TIGAR Attenuates High Glucose-Induced Neuronal Apoptosis via an Autophagy Pathway”, <doi: https://doi.org/10.3389/fnmol.2019.00193>).

Currently, the techniques developed to treat a stroke are very rapid intervention, within 4 hours to 5 hours following the stroke and elimination of the clot chemically via a recombinant tissue plasminogen activator, rtPA, or mechanically, by thrombectomy to allow reperfusion. However, these two ways of intervening in the face of a stroke do not exhibit optimal efficacy and are subjected to quite restrictive patient inclusive criteria.

A composition comprising, in a pharmaceutically acceptable carrier, at least one compound of formula (I) for preventing loss of hearing in a subject or for obtaining at least partial restoration of the hearing of a treated subject having, before treatment, a reduced auditory function is known from document WO 2016/016518 A2.

The effect of Ganoderma total sterol (GS) and the main components thereof (GS1) on cultures of rat cortical neurons exposed to hypoxia/reoxygenation is also known from the document by Zhao H-B et al. (“Ganoderma total sterol (GS) and GS1 protect rat cerebral cortical neurons from hypoxia/reoxygenation injury”, <doi:https://doi.org/10.1016/j.Ifs.2004.08.013>).

A means for combating hearing loss by targeting cholesterol homeostasis is further known from the document Brigitte Malgrange et al. (“Targeting Cholesterol Homeostasis to Fight Hearing Loss: A New Perspective”, <doi: 10.3389/fnagi.2015.00003>). In addition, it is indicated that the role of cholesterol and its metabolites is not clear.

The abovementioned three documents neither disclose nor suggest a composition comprising at least one compound of formula (I) for use thereof in the treatment of a neuronal pathology of a subject, said neuronal pathology being related to hypoxia and/or to hypoglycemia affecting cells of the central nervous system.

There is therefore a need to develop compounds which make it possible to treat patients having been subjected to oxygen and/or glucose depravation, caused for example by cerebral ischemia, hypoglycemia or hypoxia, in order to improve their recovery.

### SUMMARY

One idea forming the basis of the invention is to provide preventive and/or curative compositions that are of use for the treatment of neurological diseases, affecting in particular the neurons of the central nervous system, involving hypoxia, hypoglycemia and/or hyperglycemia.

For that, the invention provides a composition comprising at least one compound of formula (I):

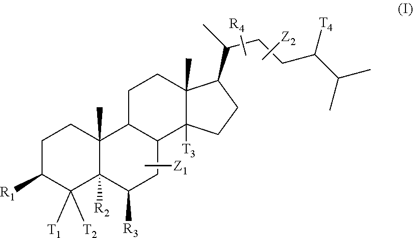

formula wherein
R₁═OH, F, OCₙH₂ₙ₊₁, R—COO, R—OCOO, RHN—COO or OPO(OR)₂ with R═H or CₙH₂ₙ₊₁ with n≤16;

### R₂═H or OH;

R₃=—NR₅R₆,
R₅ being H or —(CH₂)₃NH₂, and
R₆ being taken from the group formed by —(CH₂)₃NH(CH₂)₄NHR₇; —(CH₂)₄NH(CH₂)₃NHR₇; —(CH₂)₃NH(CH₂)₄NH(CH₂)₃NHR₇; —(CH₂)₃NHR₇; —(CH₂)₄NHR₇ with R₇═H or COCH₃; —(CH₂)₂-imidazol-4-yl; —(CH₂)₂-indol-3-yl; and
R₄═H or OH in position 20, 22, 24, 25, 26 or 27, positioned so as to create an asymmetric center of configuration R or S;
Z₁ and Z₂ each represent the number of double bonds between the carbon atoms C7 and C8 and C22 and C23 respectively (either 0 or 1); T₁, T₂ and T₃=H or CH₃ independently of one another; T₄=H, CH₃, C₂H₅ positioned so as to obtain an asymmetric center of configuration R or S in position 24;
and/or at least one pharmaceutically acceptable salt of at least one compound of formula (I),
for use thereof in the treatment of a neuronal pathology of a subject, said neuronal pathology being related to hypoxia, to hypoglycemia and/or to hyperglycemia affecting cells of the central nervous system.

The compound of formula (I) and defined by: Z₁=Z₂=0; R₁=R₂═OH; R₄═H; R₅═H; R₆=—(CH₂)₃—NCOOC(CH₃)₃—(CH₂)₄—NHCOOC(CH₃)₃; T₁=T₂=T₃=T₄=H is called DX243BOC, illustrated in table 1.

The COOC(CH₃)₃ substituent is also called tert-butoxycarbonyl or Boc functional group.

The compound of formula (I) belongs to the steroid group. The numbering of the carbon atoms of the compound of formula (I) thus follows the nomenclature defined by the IUPAC in Pure & Appl. Chem., Vol. 61, No. 10, pp. 1783-1822, 1989. The numbering of the carbon atoms of a compound belonging to the steroid group according to the IUPAC is illustrated below:

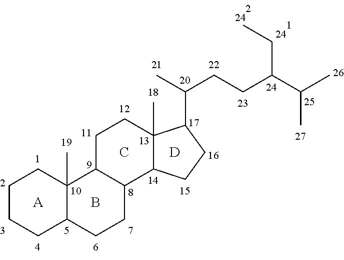

The methods for preparing the compound of formula (I) have already been described beforehand, and in particular in DE MEDINA, P. et al., Synthesis of New Alkylaminooxysterols with Potent Cell Differentiating Activities: Identification of Leads for the Treatment of Cancer and Neurodegenerative Diseases. Journal of Medicinal Chemistry, 52(23), 2009, pp. 7765-7777.

In addition, the composition can have one or more characteristics below, considered independently or in combination.

According to one embodiment, the compound of formula (I) is defined by Z₂=0; R₁═R₂═OH; R₄═H; R₅═H; and T₁=T₂=T₃=T₄=H.

According to one embodiment, the compound of formula (I) is defined by Z₁=0 and R₅═H.

According to one embodiment, the compound of formula (I) is defined by R₆=—(CH₂)₄NH(CH₂)₃NHR₇ with R₇═COCH₃.

According to one embodiment, the compound of formula (I) is defined by R₆=—(CH₂)₂-imidazol-4-yl.

According to one embodiment, the compound of formula (I) is defined by R₆=—(CH₂)₃NH(CH₂)₄NHR₇, —(CH₂)₄NH(CH₂)₃NHR₇, —(CH₂)₃NH(CH₂)₄NH(CH₂)₃NHR₇; or —(CH₂)₄NHR₇; and R₇═H.

According to one embodiment, the compound of formula (I) is defined by Z₁=1 and R₅═H.

According to one embodiment, the compound of formula (I) is defined by:
R₁═F, OCₙH₂ₙ₊₁, R—COO, R—OCOO, RHN—COO or OPO(OR)₂ with R═H or CₙH₂ₙ₊₁, with n≤16;
R₂═OH;
R₅═H;
R₆=—(CH₂)₄NH(CH₂)₃NHR₇, —(CH₂)₃NH(CH₂)₄NHR₇, —(CH₂)₃NH(CH₂)₄NH(CH₂)₃NHR₇; —(CH₂)₄NHR₇;
Z₁=0 or Z₁=1;

### Z₂=0.

According to one embodiment, the compound of formula (I) is defined by R₆=—(CH₂)₃NH(CH₂)₄NHR₇; —(CH₂)₄NH(CH₂)₃NHR₇; or —(CH₂)₃NH(CH₂)₄NH(CH₂)₃NHR₇; and R₇═H.

According to one embodiment, the at least one compound of formula (I) is defined by Z₁=Z₂=0; R₁=R₂═OH; R₄═H; R₅═H; R₆═(CH₂)₃NH(CH₂)₄NH₂; T₁=T₂=T₃=T₄=H. In this embodiment, the compound is called DX243. The results obtained with this compound are particularly advantageous. Indeed, a curative effect of this compound against the pathophysiological phenomenon of hypoxia, hypoglycemia and/or hyperglycemia is observed in very low concentrations.

According to one embodiment, the at least one compound of formula (I) is defined by Z₁=Z₂=0; R₁=R₄═H; R₂═OH; R₅═H; R₆═(CH₂)₄NH(CH₂)₃NH₂; T₁=T₂=T₃=T₄=H. In this embodiment, the compound is called DX245. The results obtained with this compound are particularly advantageous. Indeed, a curative effect of this compound against the pathophysiological phenomenon of hypoxia, hypoglycemia and/or hyperglycemia greater than that of DX243 is observed.

According to one embodiment, the at least one compound of formula (I) is defined by Z₁₌₁; Z₂=0; R₁=R₄═H; R₂═OH; R₅═H; R₆═(CH₂)₃NH(CH₂)₄NH₂; T₁=T₂=T₃=T₄ ═H. In this embodiment, the compound is called DX242. The results obtained with this compound are particularly advantageous. Indeed, a curative effect of this compound against the pathophysiological phenomenon of hypoxia, hypoglycemia and/or hyperglycemia greater than that of DX243 is observed.

According to one embodiment, the at least one compound of formula (I) is defined by Z₁=1; Z₂=0; R₁=R₄═H; R₂═OH; R₅═H; R₆═(CH₂)₄NH(CH₂)₃NH₂; T₁=T₂=T₃=T₄ ═H. In this embodiment, the compound is called DX244. The results obtained with this compound are particularly advantageous. Indeed, a curative effect of this compound against the pathophysiological phenomenon of hypoxia, hypoglycemia and/or hyperglycemia greater than that of DX243 is observed.

According to one embodiment, the neuronal pathology of the central nervous system is taken from the group consisting of cerebral traumas and strokes.

According to one embodiment, the neuronal pathology of the central nervous system is a cerebral lesion due to ischemia.

According to one embodiment, the neuronal pathology of the central nervous system is a cerebral lesion due to respiratory insufficiency. The term “respiratory insufficiency” is intended to mean obstructive respiratory insufficiency and restrictive respiratory insufficiency. Said respiratory insufficiency may originate for example from a pulmonary infection of bacterial or viral origin, for example following a coronavirus infection.

According to one embodiment, the hypoglycemia is due to diabetes.

According to one embodiment, the hyperglycemia is due to diabetes.

According to one embodiment, the invention also provides a composition for use thereof in the treatment of a neuronal pathology of a subject, said neuronal pathology being related to hypoxia, to hypoglycemia and/or to hyperglycemia affecting cells of the central nervous system, said composition being in the form of an aqueous solution and having a concentration of compound of formula (I) of between 1 pmol·L⁻¹ and 1 mmol·L⁻¹, preferentially between 10 pmol·L⁻¹ and 0.1 mmol·L⁻¹, more preferentially between 0.1 nmol·L⁻¹ and 1 μmol·L⁻¹. For example, the concentration of compound of formula (I) is between 1 nmol·L⁻¹ and 150 nmol·L⁻¹.

### BRIEF DESCRIPTION OF THE FIGURES

The invention will be understood more clearly, and other aims, details, characteristics and advantages thereof will emerge more clearly, over the course of the following description of several particular embodiments of the invention, given solely by way of nonlimiting illustration, with reference to the appended drawings.

FIG. 1 represents schematically a protocol for studying the efficacy of compounds of formula (I), in particular of DX243, on an in vitro model of ischemia.

FIG. 2 illustrates the results of an immunocytochemical test in order to evaluate neuronal survival in an in vitro model of ischemia/reperfusion in the presence of DX243.

FIG. 3 illustrates the results of an MTT neuronal survival evaluation test in an in vitro model of ischemia/reperfusion in the presence of DX243.

FIG. 4 illustrates the results of a test for evaluating neuronal survival via trypan blue in an in vitro model of ischemia/reperfusion in the presence of DX243.

FIG. 5 illustrates the results of a test for evaluating neuronal survival via trypan blue in an in vitro model of ischemia/reperfusion in the presence of compounds of formula (I) of table 1, other than DX243.

FIG. 6 illustrates the results of an MTT neuronal survival evaluation test in an in vitro model of ischemia/reperfusion in the presence of the compounds of table 1.

FIG. 7 illustrates the results of a second test for evaluating neuronal survival via trypan blue in an in vitro model of ischemia/reperfusion in the presence of the compounds of formula (I) of table 1.

FIG. 8 illustrates the results of the in vivo stroke test in mice in order to evaluate neuronal survival after having undergone a stroke followed by reperfusion with DX243 treatment.

FIG. 9 is a set of graphs illustrating results of the in vivo stroke test in mice in order to evaluate neuronal survival after having undergone a stroke followed by reperfusion with DX243 treatment.

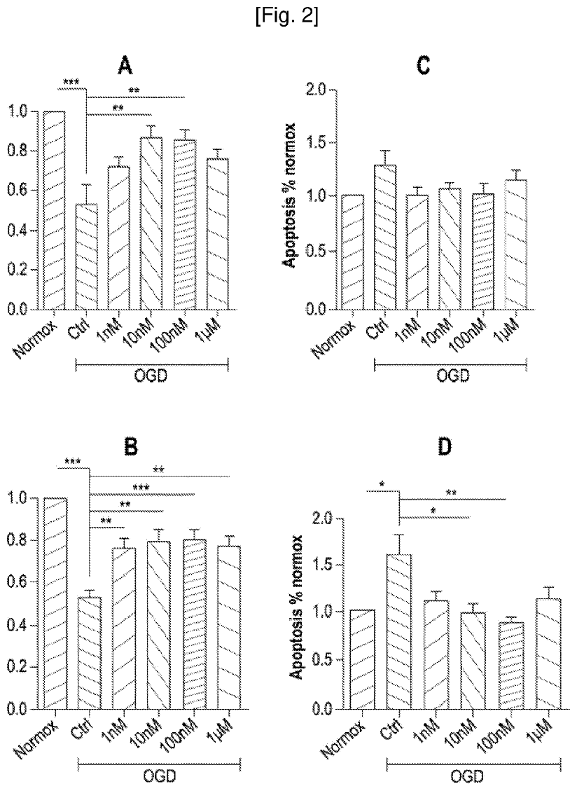

In FIGS. 2 to 4, 6 and 7, the stars indicate the statistical power of the results. A star indicates that it is 95% certain that the results are not random. The presence of 2 stars means that it is 99% certain that the results are not random, and the presence of 3 stars indicates that it is 99.9% certain that the results are not random.

### DESCRIPTION OF EMBODIMENTS

Several experimental protocols which demonstrate the protective effect of the compounds of formula (I) indicated in table 1, including the compound DX243, against hypoxia and hypoglycemia will be described below.

The concentrations or molarities of the compounds expressed in mole per liter, the symbol of which is mol·L⁻¹ or M.

### Example 1: Obtaining Cortical Neurons

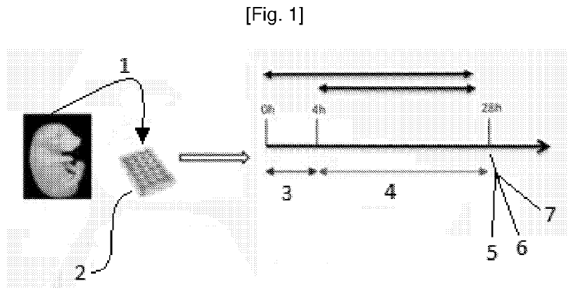

With reference to FIG. 1, the first step of the protocol for studying the neuroprotective effect of the compounds of formula (I), in particular those indicated in table 1, consists in obtaining a primary culture of cortical neurons from cells taken from wild-type mouse embryonic brains placed under appropriate culture conditions (step 1). More specifically, the cells taken are grown in a Neurobasal™ medium (ref. 21103049 ThermoFisher Scientific) to which L-glutamine and B27 supplement 50X (ref. 17504044 ThermoFisher Scientific) have been added (step 2). The neurons from the primary culture are then isolated and purified. It should be noted that it is the culture conditions in themselves which make it possible to obtain a purified culture of neurons from the dissociation of the embryonic cortexes. Oxygen and glucose deprivation (OGD) is then carried out (step 3) on these neurons in order to mimic as closely as possible what happens in vivo during a stroke, that is to say a decrease in oxygen and glucose supply due to a decrease in blood perfusion for the cells. In order to carry out this oxygen and glucose deprivation mimicking ischemia, the neurons are placed, for 4 h, in an incubator of which the atmosphere has an oxygen content of approximately 1% and with the culture medium being replaced with a glucose-free medium (step 3). Since the ischemia model is an “ischemia/reperfusion” model, the neurons are then placed in a medium containing glucose under normoxic conditions for 24 h (reperfusion) (step 4) following the ischemia step. Neuron group A is a group of neurons having been treated with a solution of compound of formula (I) during the OGD and the reperfusion, that is to say that a molecule of table 1 is added to glucose-free medium as soon as the oxygen and glucose deprivation begins (step 3). The concentration of the compound of formula (I) called DX243 is between 1 nmol·L⁻¹ and 1 μmol·L⁻¹ in the glucose-free medium. The concentration of the other molecules of table 1 is 100 nmol·L⁻¹ in the glucose-free medium. Neuron group B is a group of neurons having been treated with a solution of DX243 during the reperfusion only (step 4), in order to be closer to what occurs clinically, namely the possible treatment only after several hours with a conventional treatment. The DX243 solution is added to the glucose-free medium as soon as the reperfusion begins (step 4). The DX243 concentration is between 1 nmol·L⁻¹ and 1 μmol·L⁻¹ in the glucose-free medium.

The negative control consists of a group of neurons undergoing OGD (step 3) followed by reperfusion (step 4) without DX243 being present. This group of neurons is called “Ctrl” in FIGS. 2 to 4. The normal control consists of a group of neurons from the primary culture placed under normoxic conditions for 28 h. The normal control is called “normox” in FIGS. 2 to 4. The positive control consists of a group of neurons from the primary culture having undergone OGD for 4 h (step 3) in the presence of roscovitine, which is a cell cycle inhibitor, leading to neuronal protection, followed by reperfusion for 24 h (step 4). The positive control is called “ROSCO” in FIGS. 2 to 4.

At the end of the reperfusion (step 4), 24 h after the OGD, the survival of the neurons is evaluated by means of three different tests: an immunocytochemical test 5, an MTT (3-(4,5-dimethylthiazol-2-yl)-2,5-diphenyltetrazolium bromide) test 6 and a test based on the cell membrane integrity via the use of trypan blue 7. The results are then compared to the results of the three control-group survival tests.

### Example 2: Immunocytochemical Test for Neuronal Survival in the Case of the Use of DX243

FIG. 2 presents the results of the first neuronal survival test in the case of the use of DX243. Graphs A and C are a representation of the results obtained for group A; the y-axis represents the proportion of live cells relative to the normox group. Graphs B and D are a representation of the results obtained for group B; the y-axis represents the proportion of cells undergoing apoptosis relative to the normox group. This first test is an immunocytochemical test which makes it possible to demonstrate the live cells and the dead cells.

A first series of images is taken with chemical labelling by fluorescence. This labelling is carried out with DAPI (4′,6-diamidino-2-phenylindole) which is capable of strongly binding to the adenine (A) and thymine (T) bases of DNA. It makes it possible to detect the live cells. A second series of images is taken with labelling by fluorescence using the CC3 antibody. The CC3 antibody makes it possible to detect activated caspase 3, and therefore the cells in a state of apoptosis.

In order to visualize the proportion of healthy neurons among the various culture conditions, the TUJ1 antibody is used. TUJ1 reacts with beta-tubulin Ill, a structural protein of which tubulin is made and which is specific for neurons. Beta-tubulin Ill is widely used as a marker for distinguishing neurons from other cell types.

Based on these images, the proportion of live cells (cell survival) was calculated by determining the ratio of the number of live cells to the total number of cells. The proportion of cells undergoing apoptosis was determined using the ratio of the number of cells detected by the CC3 antibody to the total number of cells. The values obtained were related to those obtained by the normox group.

It is observed that OGD induces a decrease in the proportion of live cells in the Ctrl group on graphs A and B of FIG. 2, and an increase in the proportion of cells undergoing apoptosis in the Ctrl group of graphs C and D of FIG. 2. On the other hand, both a treatment with DX243 during OGD and reperfusion and a treatment with DX243 only during reperfusion partially protect against neuronal death. Indeed, an increase in the proportion of live cells and a decrease in the proportion of cells undergoing apoptosis are observed following the treatment with DX243, compared with the Ctrl group. A DX243 concentration of between 1 nmol·L⁻¹ and 100 nmol·L⁻¹ corresponds to an ideal concentration range for maximizing the neuroprotective effects.

Furthermore, it appears that a post-OGD treatment for 24 h with DX243 protects the neurons even more efficiently.

### Example 3: MTT Neuronal Survival Evaluation Test in the Case of the Use of DX243

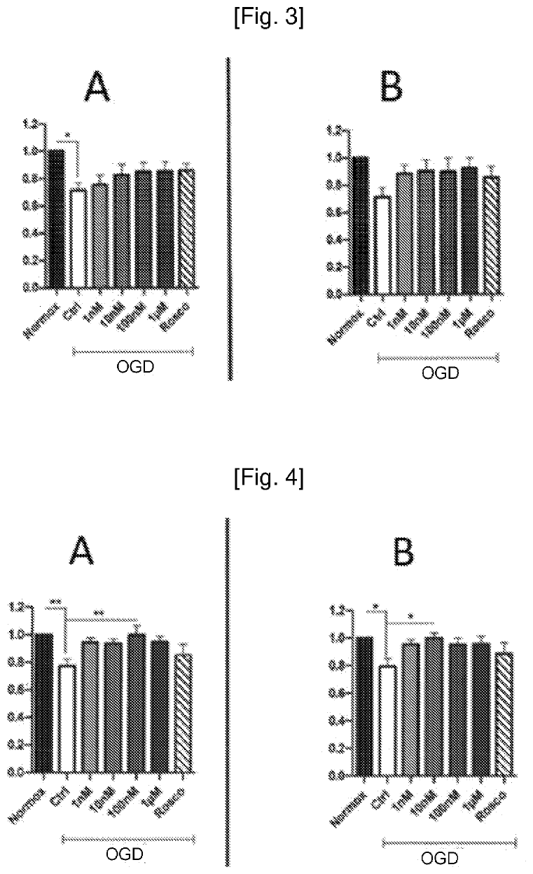

In order to confirm the results obtained with DX243, a test based on the metabolic activity of the neurons was carried out to evaluate neuronal survival in another way. This test is based on the use of the tetrazolium salt MTT (3-(4,5-dimethylthiazol-2-yl)-2,5-diphenyltetrazolium bromide). The tetrazolium is reduced, by the mitochondrial succinate dehydrogenase of the active live cells, into formazan, a purple-colored precipitate. The amount of precipitate formed is proportional to the amount of live cells but also to the metabolic activity of each cell. Thus, a simple assay of the optical density at 550 nm by spectroscopy makes it possible to determine the relative amount of live and metabolically active cells. Given that the higher the number of live cells, the greater the colorimetric intensity, the colorimetric intensity was consequently quantified and related to the intensity observed under normoxic conditions. The results obtained were reported in graph A of FIG. 3 for group A and in graph B of FIG. 3 for group B.

On graphs A and B of FIG. 3, it is observed that the OGD leads to a marked decrease of the metabolically active cells in the Ctrl group. On the other hand, treatment of the neurons with DX243 partially protects against this decrease in survival following OGD. This effect is all the more marked for the group of neurons having benefited from DX243 only during the reperfusion for 24 h, as shown by group B of FIG. 3. On the two graphs, a treatment comprising a DX243 concentration of between 10 nmol·L⁻¹ and 100 nmol·L⁻¹ appears to be the most efficacious.

### Example 4: Test for Evaluating Neuronal Survival Via Trypan Blue in the Case of the Use of DX243

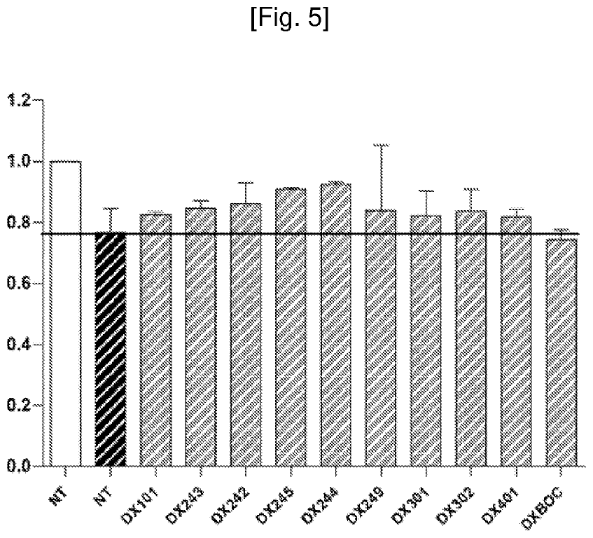

A third cell survival test in the case of the use of DX243, based on cell membrane integrity, which is ruptured in dead cells, was carried out. The latter test uses trypan blue which will stain the dead cells blue. The calculation of the percentage of live cells was consequently performed by counting the proportion of blue and non-blue cells, and related to the percentage of live cells observed in the normox group. The results obtained were reported in graph A of FIG. 4 for group A and in graph B of FIG. 4 for group B.

It is observed that the OGD induces a significant decrease in the number of cells having retained the integrity of their membrane, whether on graph A of FIG. 4 or on graph B of FIG. 4. Treatment of the neurons with DX243 restores the percentage of live cells to levels close to that observed in the normox group, mainly with concentrations between 1 nmol·L⁻¹ and 100 nmol·L⁻¹.

DX243 is therefore efficacious at concentrations between 1 nmol·L⁻¹ and 1 μmol·L⁻¹. For the DX243 concentrations between 10 nmol·L⁻¹ and 100 nmol·L⁻¹, the results are more stable and the protective effect of DX243 is statistically greater. This can be explained by the fragility of the primary cultures outside their natural environment.

### Example 5: Neuronal Survival Evaluation Test Via Trypan Blue in the Case of the Use of the Molecules of Table I

For all the molecules of table I other than DX243 used on group A, the third cell survival test was carried out. The results obtained were reported on the graph of FIG. 5, with the results obtained with DX243 being included therein. The sign NT means not treated. The non-hatched bar NT corresponds to a group of neurons derived from the primary culture, placed under normoxic conditions for 28 h. The hatched bars represent groups of neurons derived from the primary culture having undergone an OGD and reperfusion, either without molecules of table 1 (hatched bar called NT) or in the presence of a molecule of table 1. The calculation of the percentage of live cells was performed by counting the proportion of blue and non-blue cells, and related to the percentage of live cells observed in the NT group under normoxic conditions, that is to say the NT group not having undergone OGD (not represented).

For all the compounds tested, with the exception of DX243BOC, the survival rate of the groups of neurons is greater than that of the groups of NT neurons having undergone OGD and reperfusion (hatched bar NT); they therefore exhibit a neuroprotective effect. In particular, it should be noted that DX245, DX244 and DX242 have a survival rate greater than that of DX243.

The survival rate of the group treated with DX243BOC is for its part lower than that of the group treated with DX243 and than that of the NT group having undergone OGD and reperfusion, thus demonstrating the importance of the R₃ group in the activity of the compound of formula (I).

### Example 6: MTT Neuronal Survival Evaluation Test in the Case of the Use of the Molecules of Table 1

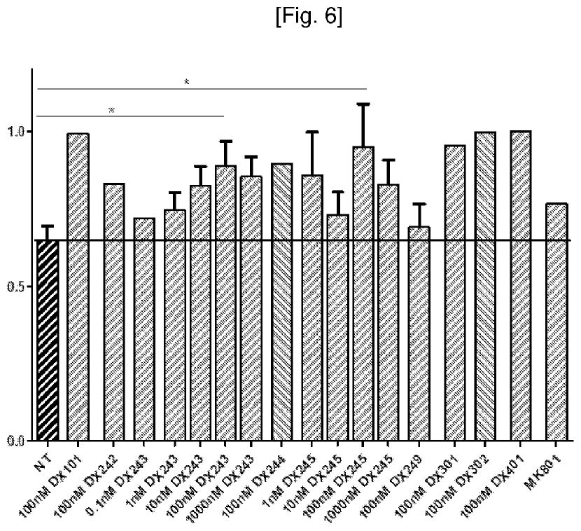

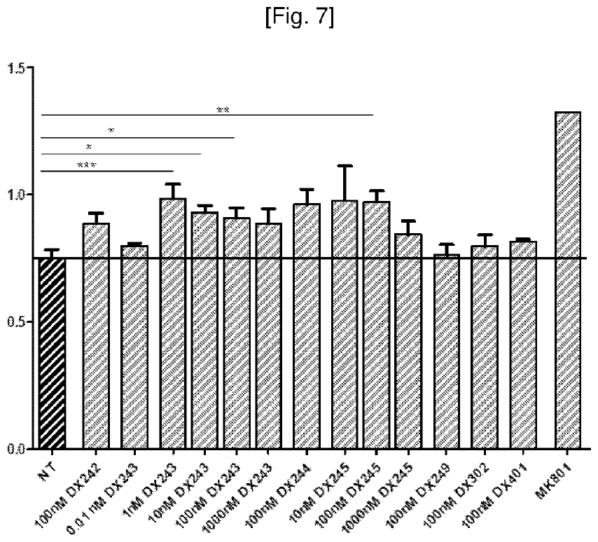

On the graph of FIG. 6, a test similar to the FIG. 3 graph A table was carried out, that is to say an MTT test in order to evaluate the neuroprotective effect of the molecules after having undergone treatment for a period of 4 h of OGD and then 24 h of reperfusion. The test is also based on the protocol for obtaining cortical neurons of example 1 given above. Unlike the example of FIG. 3A, the roscovitine positive control was replaced with a dizocilpine (MK801) positive control. This positive control also consists of a group of neurons derived from the primary culture having undergone OGD for 4 h in the presence of dizocilpine, followed by reperfusion for 24 h. The positive control is called “MK801” in FIGS. 6 and 7. The y-axis of FIGS. 6 and 7 illustrates as a percentage the ratio of the treated live cells with regard to the live cells under normoxic conditions, not represented in the graph since it is equal to 100%. MK801 is a noncompetitive N-methyl-D-aspartate (NMDA) receptor antagonist and is known to have a neuroprotective effect. All the compounds of table 1 were tested. In FIG. 6, a significant neuronal protection is observed with a treatment at 100 nM of DX243 and DX245. A neuroprotective effect is observed with this test for DX101, DX242, DX244, DX301, DX302, DX401, DX249.

For all the compounds tested in this example, the survival rate of the groups of neurons is greater than that of the groups of NT neurons; they therefore exhibit a neuroprotective effect.

### Example 7: Neuronal Survival Evaluation Test Via Trypan Blue in the Case of the Use of the Molecules of Table 1

A further cell survival test for the various compounds of table 1 was carried out and the results are presented in FIG. 7. This test is similar to that carried out in FIG. 4. It is based on the cell membrane integrity, which is ruptured in dead cells. This test uses trypan blue which will stain the dead cells blue. It is carried out in a manner similar to the test of FIG. 4A, that is to say based on the protocol for obtaining cortical neurons of example 1 given above. Unlike the example of FIG. 4A, the roscovitine positive control was replaced with the dizocilpine (MK801) positive control. The aim of this test is to evaluate the neuroprotective effect of the molecules after having undergone a treatment for a period of 4 h of OGD and then 24 h of reperfusion, that is to say a total of 28 h of experiment. All the compounds of table 1 were tested several times. In addition, DX243 and DX245 were tested in various amounts indicated in FIG. 7.

At the end of this experiment, a significant neuronal protection is observed with a treatment at 100 nM of DX245 and for the concentrations ranging from 1 to 100 nM of DX243. A neuroprotective effect is also observed with a test for 10 nM of DX245, 1000 nM of DX245, 1000 nM of DX243, DX242 and 244.

It is therefore expected that, in a pharmaceutically acceptable aqueous solution comprising the compound of formula (I), a concentration of compound of formula (I) of between 1 μmol·L⁻¹ and 1 mmol·L⁻¹, preferentially between 10 μmol·L⁻¹ and 0.1 mmol·L⁻¹, more preferentially between 0.1 nmol·L⁻¹ and 1 μmol·L⁻¹, will be efficacious.

### Example 8: Study of the Neuroprotective Effect In Vivo in a Murine Model

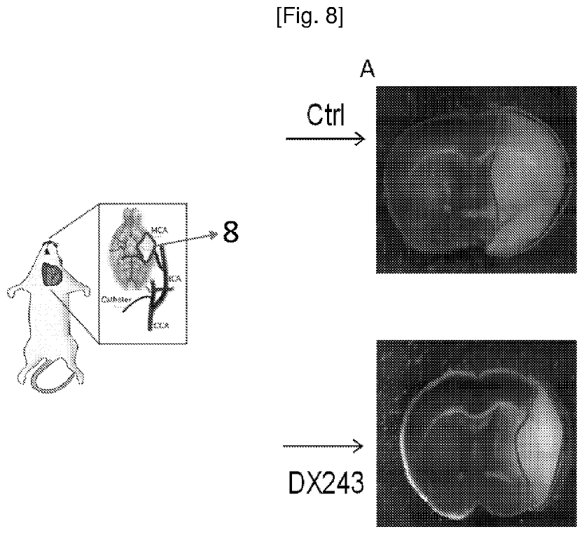

A study was carried out in vivo in order to demonstrate the neuroprotective effect of the DX243 molecule in a male murine model of stroke, called Middle Cerebral Artery Occlusion (MCAO), represented in FIG. 8, said male mice weighing approximately 20.5 grams (g) to 22 g. This in vivo model conventionally used to study the effect of molecules on stroke consists of the placing of a catheter in the anterior cingulate cortex (ACC), inserting a filament therein at the level of the common carotid artery (CCA) and pushing the filament through the common carotid artery, passing through the internal carotid artery (ICA) as far as the middle cerebral artery (MCA) leading to a considerable decrease (>80%) in blood flow in the cerebral area 8 irrigated by this artery (Estelle Rousselet et al., Modèle murin d'MCAO intraluminale: Évaluation infarctus cérébral par coloration au violet de crésyl [Murine model of intraluminal MCAO: evaluation of cerebral infarction by cresyl violet staining], Journal of Visualized Experiments and Carl Zeiss). After a period of 1 h, the filament is withdrawn so as to allow reperfusion and addition of the DX243 treatment. The DX243 injected is diluted in an aqueous solution and is at a concentration of 50 mg/Kg. The consequences are observed at 24 h after the insertion of the filament as far as the middle cerebral artery, regardless of whether said consequences are tissue consequences (triphenyltetrazolium chloride (TTC) labelling the living tissue in red) or behavioral consequences (neuroscore). The neuroscore is an evaluation of the neurological deficits of the mouse, allowing evaluation of the success of the MCAO. The scale used comprises 5 points:

0: normal state
1: slight encircling behavior with or without incoherent rotation when it is grasped by the tail, <50% of attempts at rotation towards the contralateral side.
2: slight and constant circling, >50% of attempts at rotation toward the contralateral side.
3: performs regular, strong and immediate circles, the mouse maintains a position of rotation for more than 1 to 2 seconds, its nose almost reaching its tail.
4: severe rotation progressing to rolling over, loss of walking reflex or of righting reflex.
5: comatose or moribund.

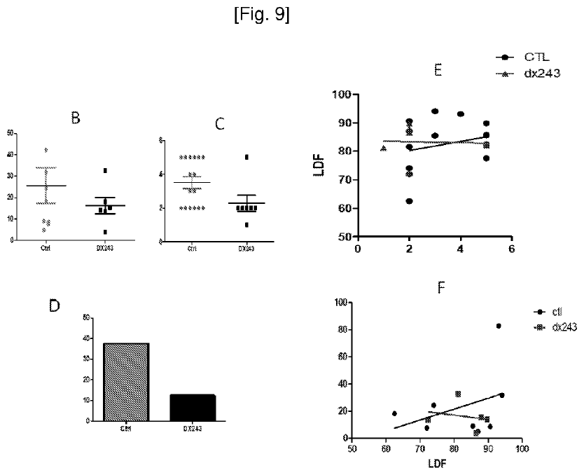

The in vivo results of the neuroprotective effect of DX243 are presented in FIGS. 8 and 9. Two images of TTC staining are observed in FIG. 8 (A), showing in white the area of dead tissue and representing the decrease in the infarcted area in the mice treated with DX243. Graph B of FIG. 9 represents the decrease in the percentage of infarcted area between treated or non-treated (Ctrl) mice; indicated along the y-axis is the percentage of the infarcted region with regard to the total size of the brain of the mouse. Graph C illustrates the behavioral improvement (neuroscore) in the treated mice. Graph D represents the decrease in the percentage of dead mice in the mice treated with DX243. Graphs E and F represent the disappearance in correlation between decrease in blood flow and histological and behavioral consequences following treatment with DX243. The decrease in the blood flow was measured by Laser Doppler Flowmetry (LDF). It has been demonstrated in FIG. 9 that treatment with DX243 makes it possible to improve, in 24 h, the consequences of a stroke in the mouse. Indeed, in the group of mice treated with DX243, a decrease in the percentage of dead mice is seen on graph D. Graph B likewise illustrates a decrease in the size of the lesion (infarcted region) and graph C illustrates a decrease in the behavioral deficits (neuroscore) in the living mice. In addition, it is observed, via a laser doppler, the results of which are presented on graphs E and F, that the greater the decrease in blood flow, the more significant the deficits. On the other hand, in the treated mice, there does not appear to be any correlation between the decrease in blood flow and the consequences of the stroke. These in vivo results demonstrate the observed neuroprotective effect of the molecules according to the invention.

All of these results indicate that the compounds DX101, DX243, DX242, DX245, DX244, DX249, DX301, DX302 and DX401 of table 1 have a neuroprotective effect against cerebral ischemia, and therefore against neuronal pathologies related to hypoxia, to hypoglycemia and/or to hyperglycemia, such as cerebral traumas and strokes.

Although the experiments were carried out with ten different compounds of formula (I), it is clearly obvious that similar results are expected for the compounds of formula (I) other than those of table 1.

In addition, in the context of hypoxia alone or of hypoglycemia alone, an apoptotic neuronal death component is observed, as described in Kato, et al. (“Recurrent short-term hypoglycemia and hyperglycemia induce apoptosis and oxidative stress via the ER stress response in immortalized adult mouse Schwann (INMS32) cells”. Neuroscience Research, 13 Nov. 2018, retrieved from <https://www.sciencedirect.com/science/article/pii/S0168010218304371?via%3Dihub> <doi:https://doi.org/10.1016/j.neures.2018.11.004>), in Sendoel A., et al. (“Apoptotic Cell Death Under Hypoxia”. Physiology, 29, pp. 168-176, 2014), or else in Xu, Y. et al. (“Protective effect of lithium chloride against hypoglycemia-induced apoptosis in neuronal PC12 cell”. Neuroscience, 330, 25 Aug. 2016, pp. 100-108). Thus, the results observed above in the ischemia/reperfusion model show protection due to the compounds of formula (I) in the face of this apoptotic component. Furthermore, hypoglycemia is capable of inducing oxidative stress in neuronal cells, which oxidative stress is present in the context of oxygen and glucose deprivation and which is a major component of the induction of neuronal death. Consequently, common mechanisms which induce neuronal death exist in the context of neuronal death induced by hypoglycemia and hypoxia, by hypoglycemia alone and by hypoxia alone, such as apoptosis. In conclusion, the neuroprotective effect of the compounds of formula (I) according to the invention extends to cases of hypoxia alone or of hypoglycemia alone.

The use of the verbs “contain”, “comprise” or “include” and of conjugated forms thereof does not exclude the presence of elements or steps other than those stated in a claim.

In the claims, any reference sign between parenthesis cannot be interpreted as a limitation of the claim.

TABLE 1

| Name | Structure |
|---|---|
| DX101 | 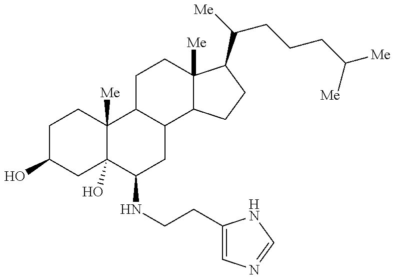 [SMILES: CC(C)CCCC(C)[C@H]1CCC2C3C[C@@H](NCCc4cnc[nH]4)[C@@]4(O)C[C@@H](O)CC[C@]4(C)C3CC[C@@]21C] |
| DX243 | 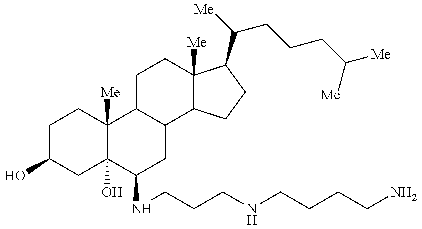 [SMILES: CC(C)CCCC(C)[C@H]1CCC2C3C[C@@H](NCCCNCCCCN)[C@@]4(O)C[C@@H](O)CC[C@]4(C)C3CC[C@@]21C] |
| DX242 | 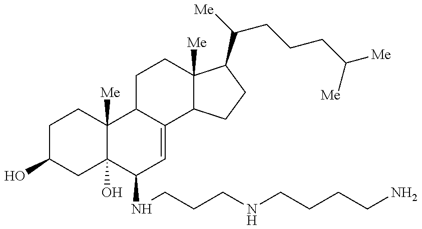 [SMILES: CC(C)CCCC(C)[C@H]1CCC2C3=C[C@@H](NCCCNCCCCN)[C@@]4(O)C[C@@H](O)CC[C@]4(C)C3CC[C@@]21C] |
| DX245 | 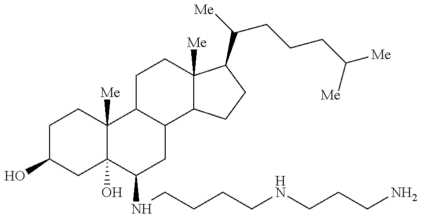 [SMILES: CC(C)CCCC(C)[C@H]1CCC2C3C[C@@H](NCCCCNCCCN)[C@@]4(O)C[C@@H](O)CC[C@]4(C)C3CC[C@@]21C] |
| DX244 | 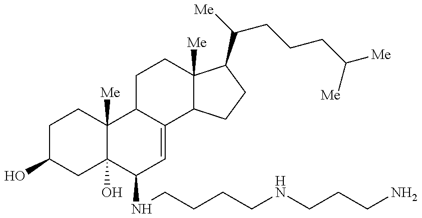 [SMILES: CC(C)CCCC(C)[C@H]1CCC2C3=C[C@@H](NCCCCNCCCN)[C@@]4(O)C[C@@H](O)CC[C@]4(C)C3CC[C@@]21C] |
| DX249 | 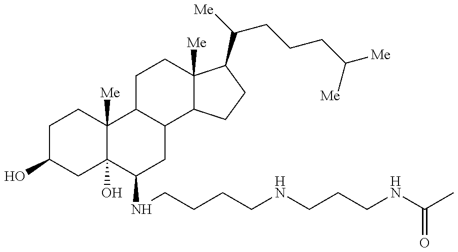 [SMILES: CC(=O)NCCCNCCCCN[C@@H]1CC2C3CC[C@H](C(C)CCCC(C)C)[C@@]3(C)CCC2[C@@]2(C)CC[C@H](O)C[C@]12O] |
| DX301 | 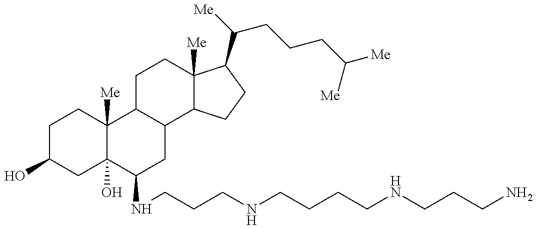 [SMILES: CC(C)CCCC(C)[C@H]1CCC2C3C[C@@H](NCCCNCCCCNCCCN)[C@@]4(O)C[C@@H](O)CC[C@]4(C)C3CC[C@@]21C] |
| DX302 | 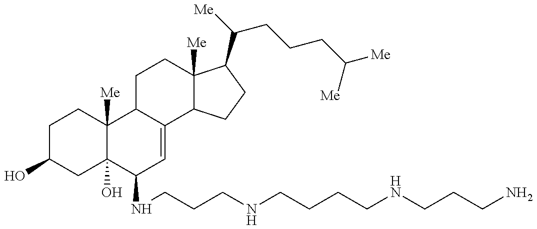 [SMILES: CC(C)CCCC(C)[C@H]1CCC2C3=C[C@@H](NCCCNCCCCNCCCN)[C@@]4(O)C[C@@H](O)CC[C@]4(C)C3CC[C@@]21C] |
| DX401 | 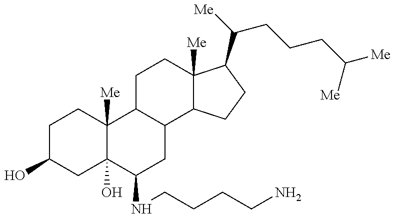 [SMILES: CC(C)CCCC(C)[C@H]1CCC2C3C[C@@H](NCCCCN)[C@@]4(O)C[C@@H](O)CC[C@]4(C)C3CC[C@@]21C] |
| DX243BOC | 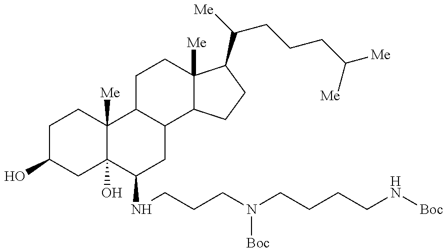 [SMILES: CC(C)CCCC(C)[C@H]1CCC2C3C[C@@H](NCCCN(CCCCNC(=O)OC(C)(C)C)C(=O)OC(C)(C)C)[C@@]4(O)C[C@@H](O)CC[C@]4(C)C3CC[C@@]21C] |

1: A composition comprising at least one compound of formula (I): 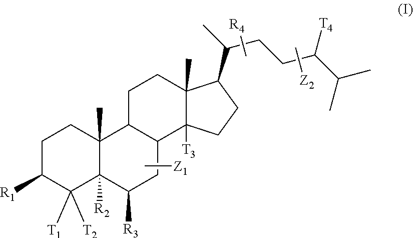
wherein;
R₁═OH, F, OCₙH₂ₙ₊₁, R—COO, R—OCOO, RHN—COO or OPO(OR)₂ with R═H or CₙH₂ₙ₊₁ with n≤16;
R₂═H or OH;
R₃=—NR₅R₆,
R₅ being H or —(CH₂)₃NH₂, and
R₆ being taken from the group formed by —(CH₂)₃NH(CH₂)₄NHR₇; —(CH₂)₄NH(CH₂)₃NHR₇; —(CH₂)₃NH(CH₂)₄NH(CH₂)₃NHR₇; —(CH₂)₃NHR₇; —(CH₂)₄NHR₇ with R₇═H or COCH₃; —(CH₂)₂-imidazol-4-yl; —(CH₂)₂-indol-3-yl; and R₄═H or OH in position 20, 22, 24, 25, 26 or 27, positioned so as to create an asymmetric center of configuration R or S;
Z₁ and Z₂ each represent the number of double bonds between the carbon atoms C7 and C8 and C22 and C23 respectively (either 0 or 1); T₁, T₂ and T₃=H or CH₃ independently of one another; T₄=H, CH₃, C₂H₅ positioned so as to obtain an asymmetric center of configuration R or S in position 24;
and/or at least one pharmaceutically acceptable salt of at least one compound of formula (I),
for use thereof in the treatment of a neuronal pathology of a subject, said neuronal pathology being related to hypoxia, to hypoglycemia and/or to hyperglycemia affecting cells of the central nervous system.

2: The composition as claimed in claim 1, wherein the compound of formula (I) is defined by Z₂=0; R₁=R₂═OH R₄═H; R₅═H; and T₁=T₂=T₃=T₄=H.

3: The composition as claimed in claim 2, wherein the compound of formula (I) is defined by Z₁=0 and R₅═H.

4: The composition as claimed in claim 3, wherein the compound of formula (I) is defined by R₆=—(CH₂)₄NH(CH₂)₃NHR₇ with R₇═COCH₃.

5: The composition as claimed in claim 3, wherein the compound of formula (I) is defined by R₆=—(CH₂)₂-imidazol-4-yl.

6: The composition as claimed in claim 3, wherein the compound of formula (I) is defined by R₆=—(CH₂)₃NH(CH₂)₄NHR₇, —(CH₂)₄NH(CH₂)₃NHR₇, —(CH₂)₃NH(CH₂)₄NH(CH₂)₃NHR₇; or —(CH₂)₄NHR₇; and R₇═H.

7: The composition as claimed in claim 2, wherein the compound of formula (I) is defined by Z₁=1 and R₅═H.

8: The composition as claimed in claim 7, wherein the compound of formula (I) is defined by R₆=—(CH₂)₃NH(CH₂)₄NHR₇; —(CH₂)₄NH(CH₂)₃NHR₇; or —(CH₂)₃NH(CH₂)₄NH(CH₂)₃NHR₇; and R₇═H.

9: The composition as claimed in claim 1, wherein the compound of formula (I) is defined by:
R₁═F, OCₙH₂ₙ₊₁, R—COO, R—OCOO, RHN—COO or OPO(OR)₂ with R═H or CₙH₂ₙ₊₁, with n≤16;
R₂═OH;
R₅═H;
R₆=—(CH₂)₄NH(CH₂)₃NHR₇, —(CH₂)₃NH(CH₂)₄NHR₇, —(CH₂)₃NH(CH₂)₄NH(CH₂)₃NHR₇; —(CH₂)₄NHR₇;
Z₁=0 or Z₁=1
Z₂=0.

10: The composition as claimed in claim 1, wherein the neuronal pathology of the central nervous system is chosen from the group consisting of cerebral traumas and strokes.

11: The composition claim 1, wherein the neuronal pathology of the central nervous system is a cerebral lesion due to ischemia.

12: The composition as claimed in claim 1, wherein the neuronal pathology of the central nervous system is a cerebral lesion due to respiratory insufficiency.

13: The composition as claimed in claim 1, wherein the hypoglycemia is due to diabetes.

14: The composition as claimed in claim 1, wherein the hyperglycemia is due to diabetes.

14: The composition as claimed in claim 1, in the form of an aqueous solution and having a concentration of compound of formula (I) of between 1 μmol·L⁻¹ and 1 mmol·L⁻¹.
